SEQUENCE LISTING (5 sequences)

SEQ ID NO: 1 - DNA, 20 bp, Artificial Sequence
  Description of Artificial Sequence Synthetic oligonucleotide
gaaaatgtttcctgactcag

SEQ ID NO: 2 - DNA, 20 bp, Artificial Sequence
  Description of Artificial Sequence Synthetic oligonucleotide
ccccggacgatattgaacaa

SEQ ID NO: 3 - DNA, 19 bp, Artificial Sequence
  Description of Artificial Sequence Synthetic oligonucleotide
tcctacacaatgcgttgcc

SEQ ID NO: 4 - DNA, 20 bp, Artificial Sequence
  Description of Artificial Sequence Synthetic oligonucleotide
tggcacactgtgagctgcct

SEQ ID NO: 5 - DNA, 20 bp, Artificial Sequence
  Description of Artificial Sequence Synthetic oligonucleotide
gcccctagaaggtggctacc

(19) United States
(12) Patent Application Publication
(10) Pub. No.: US 2022/0025332 A1
(43) Pub. Date: Jan. 27, 2022

(54) THERAPEUTIC USES OF GENE EDITED FIBROBLASTS

(71) Applicant: Figene, LLC, Houston, TX (US)

(72) Inventors: Pete O'HEERON, Houston, TX (US); Thomas ICHIM, San Diego, CA (US)

(21) Appl. No.: 17/309,649

(22) PCT Filed: Dec. 16, 2019
(86) PCT No.: PCT/US19/66575

§ 371 Date: Jun. 11, 2021

Related U.S. Application Data
(60) Provisional application No. 62/780,289, filed on Dec. 16, 2018.

(51) Int. Cl. C12N 5/077; A61K 35/33; C12N 15/11

(52) U.S. Cl. CPC C12N 5/0656; C12N 2510/00; C12N 15/111; A61K 35/33

(57) ABSTRACT

The present disclosure is directed to methods and compositions of engineered fibroblast cells with one or more types of modifications such that the cell has a reduced immune response either alone, in association with other immune cells, or both. In some embodiments, one or more targets are modified on the surface of the cell. In specific embodiments, engineered cells to be used in cellular transplantation therapy are modified to have reduced immunogenicity.

This application claims priority to U.S. Provisional Patent Application Ser. No. 62/780,289, filed Dec. 16, 2018, which is incorporated by reference herein in its entirety.

### TECHNICAL FIELD

Embodiments of the disclosure encompass at least the fields of molecular biology, cell biology, cell therapy, recombinant technology, and medicine.

### BACKGROUND

Transplantation of cellular therapies, particularly allotransplantation, allows for the utilization of “universal donor” approaches. The possibility of allotransplanting cells allows for utilization of cells that are optimized for therapeutic activity. For example, in the case of autologous bone marrow therapies, the cellular fraction possessing angiogenic or trophic activity decreases with age and is further compounded by co-morbidities of the patient, such as diabetes or peripheral artery disease. The possibility of utilizing allogeneic or xenogeneic cells allows for administration of a cellular product that is optimized for efficiency.

Allogeneic and xenogeneic cellular products have the disadvantage of rejection by the immune system of the recipient individual. There are two types of rejection processes that are known to occur. Direct rejection is stimulated by engagement of recipient T cell receptors with donor MHC II molecules, as is classically mediated by CD4+ T cells. Indirect rejection occurs when recipient antigen presenting cells engulf donor cells and present them on MHC I, thus stimulating CD8+ T cells. It is known that in the rejection of allogeneic cells that both the direct and indirect pathways of antigen presentation are involved in immune-mediated destruction. Current means of inhibiting cellular rejection include use of calcineurin inhibitors, such as cyclosporine, or inhibitors of mTOR, such as rapacymin and everolimus.

While cellular therapy trials have been performed using a variety of allogeneic cells, a variety of trials required the use of continued immune suppression. Whether fetal-derived stem cells, pancreatic islets, or embryonic derived tissues, the use of continual immune suppression has been utilized. Unfortunately, continual immune suppression predisposes the individual to increased risk of infections, neoplasia, and organ failure, particularly renal failure in the case of calcineurin inhibitor-containing regimens.

Thus, there is a need in the art for decreasing immunogenicity of cells that are to be utilized for therapeutic purposes. In addition, there is a need in the art for improved cellular therapies for a variety of medical conditions.

### BRIEF SUMMARY

The mammalian immune system provides a springboard for much of modern medicine through its ability to raise a specific response against undesirable targets in the body. However, there are other conditions where the immune response is undesirable, e.g. in transplantation, allergy and in the context of autoimmune disease. To address deficiencies in the art, the inventors developed a simple and effective method for the use of engineered cells in cellular therapy. A drawback of cellular therapy is the possibility of rejection when allogenic cells are utilized in a therapeutic manner, particularly when using mesenchymal stem cells. For example, although it is known that mesenchymal stem cells express lower amounts of HLA I, and very low to absent HLA II in an undifferentiated state, differentiation into tissues is known to cause upregulation of HLA molecules, which could potentially lead the tissue to reject the cellular therapy. Therefore, there is a need in the art for the development of cellular populations that are generated without the expression of rejection-associated antigens. Fibroblast-based therapy offers multiple benefits including: (i) ease of extraction from a source; (ii) low cost of reagents involved in expansion; and (iii) capacity to collect higher number of cells at the initiation of culture.

Accordingly, the present disclosure provides a cell (e.g., a population of cells) engineered to express less of one or more proteins comprising an extracellular domain, transmembrane domain, and an intracellular signaling domain, and wherein the expression and/or function of the protein in the cell has been reduced or eliminated. In some embodiments, the proteins are involved in regulating the immune response.

Embodiments of the disclosure consequently concern methods and compositions related to engineered fibroblasts that are modified to be less prone to eliciting a deleterious immune reaction in a recipient individual compared to an immune reaction when the fibroblasts were not accordingly modified.

One embodiment concerns the use of engineered fibroblast cells comprising a reduction in the expression of one or more polypeptide sequences encoded by one or more polynucleotide sequences within a given fibroblast cell. More particularly, but not exclusively, the present disclosure relates to engineering fibroblast cells to express less of one or more proteins comprising an extracellular domain, transmembrane domain, and an intracellular signaling domain, compared to fibroblast cells that have not correspondingly been engineered as such. In particular embodiments, the polypeptide sequence(s) that are reduced in expression comprise one or more immunogenic proteins, for example, as one or more human leukocyte antigens (HLA), one or more costimulatory molecules, one or more adhesion molecules, one or more polypeptides associated to increase HLA expression, and/or one or more polypeptides associated with the onset and continuous progression of fibrosis.

In certain aspects of the disclosure, an engineered fibroblast cell expresses one or more immunogenic proteins associated with a pathological immune response. Methods of manufacturing and using the engineered cells, regardless of the specific modification(s), are encompassed herein.

In specific embodiments, engineered fibroblast cells express less of one or more HLA proteins, including HLA-A, HLA-B, HLA-C, HLA-DP, HLA-DQ, HLA-DR, HLA-B27, or a combination thereof, compared to fibroblast cells that have not been modified.

In certain aspects, the engineered fibroblast cells express less of one or more costimulatory molecules, such as cluster of differentiation (CD)-40 (CD-40), CD-80, CD86, interleukin 12 (IL-12), or a combination thereof, compared to fibroblast cells that have not been modified.

In certain aspects, the engineered fibroblast cells express less of one or more adhesion molecules comprising lymphocyte function-associated antigen (LFA-1), interleukin adhesion molecule 1 (ICAM-1), platelet endothelial cell adhesion molecule (PECAM), epithelial cell adhesion molecule (EpCAM), CD11b, Vαβ3 integrin, or a combination thereof, compared to fibroblast cells that have not been modified.

In certain aspects, the engineered fibroblast cells express less of one or more proteins associated with an increase in HLA expression, such as interferon gamma receptor, stimulator of interferon genes (STING); class II, major histocompatibility complex, transactivator (CIIT); or a combination thereof, compared to fibroblast cells that have not been modified.

In certain aspects, the engineered fibroblast cells express less of one or more polypeptides associated with fibrosis, such as transforming growth factor beta (TGF-β) receptors types I, II, III, members of the SMAD family, such as SMAD1, SMAD2, SMAD3, SMAD4, SMAD5, SMAD6, SMAD7, SMAD8, SMAD9, or a combination thereof, compared to fibroblast cells that have not been modified.

In certain aspects of the disclosure, the engineered fibroblast cell expresses one or more immunogenic components associated with a pathological immune response. In specific embodiments the immunogenic components are HLA proteins such as HLA-A, HLA-B, HLA-C, HLA-DP, HLA-DQ, HLA-DR, HLA-B27, (CD)-40 (CD-40), CD-80, CD86, interleukin 12 (IL-12), lymphocyte function-associated antigen (LFA-1), interleukin adhesion molecule 1 (ICAM-1), platelet endothelial cell adhesion molecule (PECAM), epithelial cell adhesion molecule (EpCAM), CD11b, Vαβ3 integrin, proteins associated with an increase in HLA expression, such as interferon gamma receptor, stimulator of interferon genes (STING), CIIT, proteins associated with fibrosis, such as transforming growth factor beta (TGF-β) receptors types I, II, III, members of the SMAD family, such as SMAD1, SMAD2, SMAD3, SMAD4, SMAD5, SMAD6, SMAD7, SMAD8, SMAD9, or a combination thereof.

Embodiments of the disclosure include the genetic manipulation of fibroblasts to remove or reduce the expression of rejection-associated antigens following which the cells lack the capacity of producing gene products associated with non-desirable features, such as rejection or fibrosis.

In certain aspects, the fibroblast cells have an altered or reduced capacity for interaction or identification by immune cells, such as platelets, neutrophils, macrophages, eosinophils, T-cells, NK cells, dendritic cells, mast cells, and/or B cells that may elicit a pathologic immune response.

In particular embodiments of the disclosure, the engineered fibroblasts have a partial or complete reduction in the expression in one or more immunogenic components for example via a recombinant expression vector operable in eukaryotic cells, and the expression of the immunogenic components may be regulated by a constitutive promoter or an inducible promoter or a tissue-specific promoter, for example. In specific embodiments, the vector is a viral vector, such as a retrovirus, lentivirus, adenovirus, adeno-associated virus, or herpes simplex virus, or the vector is a non-viral vector, such as naked DNA, transposons, plasmid DNA, or minicircle DNA. Agents for transfection include at least the following: Fas ligand, TGF-beta, IL-4, IL-10, HLA-G, indolamine 2,3 deoxygenase (IDO), galectin family members, Galectin 3, arginase, and/or IL-20, as examples.

In specific embodiments, the engineered fibroblast cells are referred to as gene-edited fibroblast cells that have had one or more endogenous genes in the fibroblasts modified by the hand of man. In specific cases one or more endogenous genes in the engineered fibroblasts have been modified to have reduced expression of one or more endogenous genes, including modification to the gene itself and/or to another gene that regulates expression of another gene. The gene may be modified to incorporate one or more mutations, including one or more mutations that impact the endogenous activity of the corresponding gene product.

In some embodiments, the gene-edited fibroblast cells are immortalized. Immortalization may be accomplished by introduction of one or more certain genes, including human telomerase reverse transcriptase (hTERT). The process of immortalization is known in the art and may involve utilization of various vectors to introduce genes into target cells, including those based on viral vectors. Vectors including a nucleic acid can be expressed when the nucleic acid is operably linked to one or more expression control elements, such as promoters, enhancers, and so forth.

In certain embodiments, the disclosure pertains to the use of one or more agents to decrease the immune response of fibroblasts, such as in order to allow for transplantation, including at least allotransplantation of fibroblasts without rejection occurring. In at least some cases, the agent(s) are capable of reducing the expression of one or more immunogenic molecules in the fibroblasts.

In particular embodiments of the disclosure, one or more immunogenic components are expressed in universal donor fibroblasts and the expression of the immunogenic component is regulated by a constitutive promoter or an inducible promoter or a tissue-specific promoter, for example. In specific embodiments, the vector is a viral vector, such as a retrovirus, lentivirus, adenovirus, adeno-associated virus, or herpes simplex virus, or the vector is a non-viral vector, such as naked DNA or plasmid DNA or minicircle DNA, for example.

In one embodiment, disclosed is the use of one or more regulatory elements that are operably linked to one or more elements of a CRISPR system so as to drive expression of one or more elements of the CRISPR system, with the goal of manipulating DNA encoding immunogenic genes in fibroblasts in a manner that prevents fibroblasts from expressing the following immunogenic components, as examples: human leukocyte antigen (HLA)-A, HLA-B, HLA-C, HLA-DP, HLA-DQ, HLA-DR, HLA-B27, cluster of differentiation 40 (CD-40), CD80, CD86, CD11b, interleukin 12 (IL-12), lymphocyte function-associated antigen 1 (LFA-1), Interleukin adhesion molecule 1 (ICAM-1), platelet endothelial cell adhesion molecule (PECAM), epithelial cell adhesion molecule (EpCAM), V αβ 3 integrin, interferon gamma receptor, stimulator of interferon genes (STING), CIIT, transforming growth factor beta (TGF-β) receptors types I, II, III, members of the SMAD family, such as SMAD1, SMAD2, SMAD3, SMAD4, SMAD5, SMAD6, SMAD7, SMAD8, SMAD9, or a combination thereof. In one embodiment, CRISPR system elements that are combined in a single vector may be arranged in any suitable orientation, such as one element located 5′ with respect to or 3′ with respect to a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence, and a tracr sequence embedded within one or more intron sequences. In some embodiments, the CRISPR enzyme, guide sequence, tracr mate sequence, and tracr sequence are operably linked to and expressed from the same promoter.

In certain aspects, an individual is provided another therapy in addition to the fibroblast cell therapy. For example, before, during, and/or after the individual receives the fibroblast cell therapy, the individual may receive one or more antibiotics. Exemplary post-operative therapies includes steroids, Non Steroidal Anti-Inflammatory Drugs (NSAIDs) and/or simple pain killers (analgesics) as needed.

The present disclosure is directed to methods that allow for the use of a population of fibroblast cells for cellular therapy. Particular embodiments of the disclosure concern a reduction in the expression of one or more surface-expressed antigens, costimulatory proteins, and/or signaling proteins having a role in the immune response. In other embodiments, the aforementioned proteins are involved in the activation of the fibroblast cells.

Embodiments of the disclosure provide methods of reducing the severity of the immune response of particular types of fibroblast cells described herein. The fibroblast cells may be derived from various tissues or organs, such as skin, heart, blood vessels, bone marrow, skeletal muscle, liver, pancreas, brain, foreskin, omentum, adipose tissue, placenta, umbilical cord, which can be obtained by biopsy (where appropriate) or upon autopsy. In some aspects, the cells comprise fibroblasts, which can be from a fetal, neonatal, adult origin, or a combination thereof. In some aspects the fibroblasts are derived from mammals such as human, primate, porcine, bovine, murine, canine, and/or feline.

In some embodiments, the fibroblasts are grown in a cell culture for the purpose of stimulating fibroblast proliferation in the cell culture. In a situation of expansion of fibroblast cells, a population of fibroblast cells is subjected to one or more compositions comprised of one or more particular media and/or one or more agents such that the composition(s) are capable of reducing the immunogenicity of the population of cells. In particular embodiments of the disclosure, methods are directed to a population of cells wherein the cells are fibroblasts of any type and the fibroblasts have been modified such that they have reduced immunogenicity and may be utilized in a therapeutic capacity.

In some embodiments, the fibroblasts are grown in a cell culture in which one or more antigens that are capable of stimulating fibroblast proliferation are added to the cell culture. In a situation of expansion of fibroblast cells, a population of fibroblast cells may not be subjected to one or more compositions comprised of one or more particular media and/or one or more agents such that the composition(s) are capable of reducing the immunogenicity of the population of cells as a result of the absence of one or more particular compositions in the media.

Various quality control means are known in the art for practitioners of the disclosure to perform clinical administration of the cells. Examples of criteria for qualification of the cells includes marker identification using means such as flow cytometry, viability, endotoxin content, as well as assessment for microbial and mycoplasma contamination.

In one embodiment of the disclosure, effective amounts of the engineered fibroblasts as prepared in methods encompassed by the disclosure are administered to an individual that has or is at risk of developing medical conditions known to be or associated with inflammatory or autoimmune diseases, such as Acute Disseminated Encephalomyelitis, Acute necrotizing hemorrhagic leukoencephalitis, Addison's disease, adhesive capsulitis, Agammaglobulinemia, Alopecia areata, Amyloidosis, Ankylosing spondylitis, Anti-GBM nephritis, Antiphospholipid syndrome (APS), Anti-TBM nephritis, arthofibrosis, atrial fibrosis, autoimmune angioedema, autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune neutropenia, autoimmune oophoritis, autoimmune pancreatitis, autoimmune retinopathy, autoimmune thrombocytopenic purpura (ATP), autoimmune thyroid disease, autoimmune urticarial, axonal and neuronal neuropathies, Balo disease, Behcet's disease, benign mucosal pemphigold, bullous pemphigoid, cardiomyopathy, Castleman disease, Celiac disease, Chagas disease, chronic fatigue syndrome, Chronic inflammatory demyelinating polyneuropathy (CIDP), chronic Lyme disease, Chronic recurrent multifocal ostomyelitis (CRMO), Churg-Strauss syndrome, cicatricial pemphigold, cirrhosis, Cogans syndrome, cold agglutinin disease, congenital heart block, Coxsackie myocarditis, CREST disease, Crohn's disease, Cystic Fibrosis, deficiency of the interleukin-1 receptor antagonist, demyelinating neuropathies, dermatitis herpetiformis, dermatomyosis, Devic's disease (neuromyelitis optica), discoid lupus, Dressler's syndrome, Dupuytren's contracture, endometriosis, endomyocardial fibrosis, eosinophilic esophagitis, eosinophilic facsciitis, erythema nodosum, essential mixed cryoglobulinemia, Evans syndrome, experimental allergic encephalomyelitis, Familial Mediterranean Fever, Fibromyalgia Fibrosing alveolitis, Giant cell arteritis (temporal arteritis), giant cell myocarditis, glomerulonephritis, Glomerulonephritis, Goodpasture's syndrome, Graft-versus-host disease (GVHD), granulomatosus with polyangitis, Graves' disease, Guillain-Barre syndrome, Hashimoto's encephalitis, Hashimoto's thyroiditis, hemolytic anemia, Henoch-Schonlein purpura, hepatitis, herpes gestationis, hypogammaglobulinemia, idiopathic pulmonary fibrosis, Idiopathic thrombocytopenic purpura (ITP), IgA nephropathy, IgG4-related sclerosing disease, Immunoregulatory lipoproteins, inclusion body myositis, inflammatory bowel disorders, interstitial cystitis, juvenile arthritis, Juvenile diabetes (Type 1 diabetes), juvenile myositis, Kawasaki syndrome, keloid, Lambert-Eaton syndrome, leukocytoclastic vasculitis, lichen planus, lichen sclerosis, ligneous conjunctivitis, linear IgA disease, Lupus (SLE), Lyme disease, mediastinal fibrosis, Meniere's disease, microscopic polyangitis, Mixed connective tissue disease (MCTD), Mooren's ulcer, Mucha-Habermann disease, Multiple Sclerosis (MS), Myasthenia gravis, myelofibrosis, Myositis, narcolepsy, Neonatal Onset Multisystem Inflammatory Disease, nephrogenic systemic fibrosis, Neuromyelitis optica (Devic's), neutropenia, nonalcoholic fatty liver disease, nonalcoholic steatohepatitis (NASH), ocular-cicatricial pemphigold, optic neuritis, palindromic rheumatism, paraneoplastic cerebellar degeneration, Paroxysmal nocturnal hemoglobinuria (PNH), Parry Romberg syndrome, Pars planitis (peripheral uveitis), Parsonnage-Turner syndrome, Pediatric Autoimmune Neuropsychiatric Disorders Associated with Streptococcus (PANDAS), Pemphigus, Peripheral neuropathy, Perivenous encephalomyelitis, Pernicious anemia, Peyronie's disease, POEMS syndrome, polyarteritis nodosa, polymyalgia rhematica, polymyositis, postmyocardial infarction syndrome, postpericardiotomy syndrome, primary biliary cirrhosis, primary sclerosing cholangitis, progesterone dermatitis, progressive massive fibrosis, psoriasis, psoriatic arthritis, pure red cell aplasia, pyoderma gangrenosum, Raynauds phenomenon, reactic arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm and testicular autoimmunity, stiff person syndrome, subacute bacterial endocarditis, Susac's syndrome, sympathetic ophthalmia, systemic lupus erythematosus (SLE), Takayasu's arteritis, temporal arteritis, Thrombocytopenic purpura (TTP), Tolosa-Hunt syndrome, transverse myelitis, Tumor Necrosis Factor Receptor-associated Periodic Syndrome, Type 1 diabetes, Type I autoimmune polyglandular syndrome, Type II autoimmune polyglandular syndrome, Type III autoimmune polyglandular syndrome, ulcerative colitis, undifferentiated connective tissue disease, uveitis, vasculitis, vesiculobullous dermatosis, Vitiligo, and/or Granulomatosis with Polyangitis (GPA).

Embodiments of the disclosure are directed to a pharmaceutical composition of the fibroblast cells comprising a reduction in one or more polypeptide sequences wherein the polypeptide plays a role in the immune response as an expressed protein residing on or in a fibroblast cell. In particular cases, but not exclusively, the protein comprises an extracellular domain, a transmembrane domain, and intracellular signaling domain, or a combination thereof.

Specific embodiments of the disclosure are directed to methods of producing the fibroblast cells disclosed herein to be used in a fibroblast therapy, wherein the therapy is an immunomodulatory therapy.

Embodiments of the disclosure include an engineered fibroblast cell (or plurality thereof) comprising a reduction in the expression of at least one polynucleotide sequence encoding an immunogenic component selected from the group consisting of a) a human leukocyte antigen (HLA); b) a costimulatory molecule (cluster of differentiation 40 (CD-40), CD80, CD86, interleukin 12 (IL-12), or a combination thereof, for example); c) an adhesion molecule; d) a polypeptide associated with an increase in the expression of human leukocyte antigens; e) a polypeptide associated with fibrosis; and f) a combination thereof. The HLA may be HLA-A, HLA-B, HLA-C, HLA-DP, HLA-DQ, HLA-DR, HLA-B27, or a combination thereof. In at least some cases, the adhesion molecule comprises lymphocyte function-associated antigen 1 (LFA-1), Interleukin adhesion molecule 1 (ICAM-1), platelet endothelial cell adhesion molecule (PECAM), epithelial cell adhesion molecule (EpCAM), CD11b, V αβ 3 integrin, or a combination thereof. The polypeptide associated with an increase in the expression of HLA may comprise interferon gamma receptor, stimulator of interferon genes (STING), CIIT, or a combination thereof, for example. Examples of a polypeptide associated with fibrosis may be transforming growth factor beta (TGF-β) receptors, members of the SMAD family, or a combination thereof, for example. The reduction in the expression of the polypeptide may be mediated by any suitable means, including by CRISPR/Cas9, adenovirus, lentivirus, and/or adeno-associated virus and/or a combination thereof, for example. In particular embodiments of the disclosure any of the cells express human telomerase reverse transcriptase (hTERT). Any engineered fibroblast cells may originate from any kind of mammalian tissue. Examples of mammalian tissues include placenta, umbilical cord, foreskin, skin, omentum, adipose tissue, and/or bone marrow. The mammalian tissues may be derived from a human, primate, porcine, bovine, murine, canine, and/or feline.

In certain embodiments, there are methods of reducing an immune response to a therapy, including at least a fibroblast therapy, in an individual (including a mammal), comprising the step of providing an effective amount of any of the fibroblast cells encompassed in the disclosure. A fibroblast therapy may include an immunomodulatory therapy, for example. An individual receiving a therapy includes an individual that has or is at risk for having Acute Disseminated Encephalomyelitis, Acute necrotizing hemorrhagic leukoencephalitis, Addison's disease, adhesive capsulitis, Agammaglobulinemia, Alopecia areata, Amyloidosis, Ankylosing spondylitis, Anti-GBM nephritis, Antiphospholipid syndrome (APS), Anti-TBM nephritis, arthofibrosis, atrial fibrosis, autoimmune angioedema, autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune neutropenia, autoimmune oophoritis, autoimmune pancreatitis, autoimmune retinopathy, autoimmune thrombocytopenic purpura (ATP), autoimmune thyroid disease, autoimmune urticarial, axonal and neuronal neuropathies, Balo disease, Behcet's disease, benign mucosal pemphigold, bullous pemphigoid, cardiomyopathy, Castleman disease, Celiac disease, Chagas disease, chronic fatigue syndrome, Chronic inflammatory demyelinating polyneuropathy (CIDP), chronic Lyme disease, Chronic recurrent multifocal ostomyelitis (CRMO), Churg-Strauss syndrome, cicatricial pemphigold, cirrhosis, Cogans syndrome, cold agglutinin disease, congenital heart block, Coxsackie myocarditis, CREST disease, Crohn's disease, Cystic Fibrosis, deficiency of the interleukin-1 receptor antagonist, demyelinating neuropathies, dermatitis herpetiformis, dermatomyosis, Devic's disease (neuromyelitis optica), discoid lupus, Dressler's syndrome, Dupuytren's contracture, endometriosis, endomyocardial fibrosis, eosinophilic esophagitis, eosinophilic facsciitis, erythema nodosum, essential mixed cryoglobulinemia, Evans syndrome, experimental allergic encephalomyelitis, Familial Mediterranean Fever, Fibromyalgia Fibrosing alveolitis, Giant cell arteritis (temporal arteritis), giant cell myocarditis, glomerulonephritis, Glomerulonephritis, Goodpasture's syndrome, Graft-versus-host disease (GVHD), granulomatosus with polyangitis, Graves' disease, Guillain-Barre syndrome, Hashimoto's encephalitis, Hashimoto's thyroiditis, hemolytic anemia, Henoch-Schonlein purpura, hepatitis, herpes gestationis, hypogammaglobulinemia, idiopathic pulmonary fibrosis, Idiopathic thrombocytopenic purpura (ITP), IgA nephropathy, IgG4-related sclerosing disease, Immunoregulatory lipoproteins, inclusion body myositis, inflammatory bowel disorders, interstitial cystitis, juvenile arthritis, Juvenile diabetes (Type 1 diabetes), juvenile myositis, Kawasaki syndrome, keloid, Lambert-Eaton syndrome, leukocytoclastic vasculitis, lichen planus, lichen sclerosis, ligneous conjunctivitis, linear IgA disease, Lupus (SLE), Lyme disease, mediastinal fibrosis, Meniere's disease, microscopic polyangitis, Mixed connective tissue disease (MCTD), Mooren's ulcer, Mucha-Habermann disease, Multiple Sclerosis (MS), Myasthenia gravis, myelofibrosis, Myositis, narcolepsy, Neonatal Onset Multisystem Inflammatory Disease, nephrogenic systemic fibrosis, Neuromyelitis optica (Devic's), neutropenia, nonalcoholic fatty liver disease, nonalcoholic steatohepatitis (NASH), ocular-cicatricial pemphigold, optic neuritis, palindromic rheumatism, paraneoplastic cerebellar degeneration, Paroxysmal nocturnal hemoglobinuria (PNH), Parry Romberg syndrome, Pars planitis (peripheral uveitis), Parsonnage-Turner syndrome, Pediatric Autoimmune Neuropsychiatric Disorders Associated with Streptococcus (PANDAS), Pemphigus, Peripheral neuropathy, Perivenous encephalomyelitis, Pernicious anemia, Peyronie's disease, POEMS syndrome, polyarteritis nodosa, polymyalgia rhematica, polymyositis, postmyocardial infarction syndrome, postpericardiotomy syndrome, primary biliary cirrhosis, primary sclerosing cholangitis, progesterone dermatitis, progressive massive fibrosis, psoriasis, psoriatic arthritis, pure red cell aplasia, pyoderma gangrenosum, Raynauds phenomenon, reactic arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm and testicular autoimmunity, stiff person syndrome, subacute bacterial endocarditis, Susac's syndrome, sympathetic ophthalmia, systemic lupus erythematosus (SLE), Takayasu's arteritis, temporal arteritis, Thrombocytopenic purpura (TTP), Tolosa-Hunt syndrome, transverse myelitis, Tumor Necrosis Factor Receptor-associated Periodic Syndrome, Type 1 diabetes, Type I autoimmune polyglandular syndrome, Type II autoimmune polyglandular syndrome, Type III autoimmune polyglandular syndrome, ulcerative colitis, undifferentiated connective tissue disease, uveitis, vasculitis, vesiculobullous dermatosis, Vitiligo, and/or Granulomatosis with Polyangitis (GPA). Any mammal may be the recipient of any cells encompassed herein, including a human, primate, porcine, bovine, murine, canine, and/or feline.

Embodiments of the disclosure include methods of reducing the immunogenicity of a cell population as encompassed herein, wherein the population comprises a reduction in the expression of one or more polypeptides encoded by a polynucleotide sequence. The cell population may originate from one or more types of mammalian tissues, such as tissues derived from a placenta, umbilical cord, foreskin, skin, omentum, adipose tissue, and/or bone marrow. The mammalian tissues may be obtained from a human, primate, porcine, bovine, murine, canine, and/or feline.

Any fibroblast cells may be cultured in a media, such as one that comprises Roswell Park Memorial Institute (RPMI-1640), Dublecco's Modified Essential Media (DMEM), Eagle's Modified Essential Media (EMEM), Optimem, Iscove's Media, or a combination thereof.

In the cells, a reduction in the expression of one or more polypeptides may include one or more polypeptides that are immunogenic components. Examples of immunogenic components include at least human leukocyte antigen (HLA)-A, HLA-B, HLA-C, HLA-DP, HLA-DQ, HLA-DR, HLA-B27, cluster of differentiation 40 (CD-40), CD80, CD86, CD11b, interleukin 12 (IL-12), lymphocyte function-associated antigen 1 (LFA-1), Interleukin adhesion molecule 1 (ICAM-1), platelet endothelial cell adhesion molecule (PECAM), epithelial cell adhesion molecule (EpCAM), V αβ 3 integrin, interferon gamma receptor, stimulator of interferon genes (STING), CIIT, transforming growth factor beta (TGF-β) receptors, members of the SMAD family and/or a combination thereof. The polypeptide expression may be reduced by CRISPR/Cas9, adenovirus, lentivirus, and/or adeno-associated virus and/or a combination thereof.

In certain embodiments, there is a method of treating an autoimmune or inflammatory condition in an individual comprising a therapeutically effective amount of cells of the disclosure. The cells may be immortalized fibroblast cells, for example. The cells may express hTERT. The individual may have or is at risk of having Acute Disseminated Encephalomyelitis, Acute necrotizing hemorrhagic leukoencephalitis, Addison's disease, adhesive capsulitis, Agammaglobulinemia, Alopecia areata, Amyloidosis, Ankylosing spondylitis, Anti-GBM nephritis, Antiphospholipid syndrome (APS), Anti-TBM nephritis, arthofibrosis, atrial fibrosis, autoimmune angioedema, autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune neutropenia, autoimmune oophoritis, autoimmune pancreatitis, autoimmune retinopathy, autoimmune thrombocytopenic purpura (ATP), autoimmune thyroid disease, autoimmune urticarial, axonal and neuronal neuropathies, Balo disease, Behcet's disease, benign mucosal pemphigold, bullous pemphigoid, cardiomyopathy, Castleman disease, Celiac disease, Chagas disease, chronic fatigue syndrome, Chronic inflammatory demyelinating polyneuropathy (CIDP), chronic Lyme disease, Chronic recurrent multifocal ostomyelitis (CRMO), Churg-Strauss syndrome, cicatricial pemphigold, cirrhosis, Cogans syndrome, cold agglutinin disease, congenital heart block, Coxsackie myocarditis, CREST disease, Crohn's disease, Cystic Fibrosis, deficiency of the interleukin-1 receptor antagonist, demyelinating neuropathies, dermatitis herpetiformis, dermatomyosis, Devic's disease (neuromyelitis optica), discoid lupus, Dressler's syndrome, Dupuytren's contracture, endometriosis, endomyocardial fibrosis, eosinophilic esophagitis, eosinophilic facsciitis, erythema nodosum, essential mixed cryoglobulinemia, Evans syndrome, experimental allergic encephalomyelitis, Familial Mediterranean Fever, Fibromyalgia Fibrosing alveolitis, Giant cell arteritis (temporal arteritis), giant cell myocarditis, glomerulonephritis, Glomerulonephritis, Goodpasture's syndrome, Graft-versus-host disease (GVHD), granulomatosus with polyangitis, Graves' disease, Guillain-Barre syndrome, Hashimoto's encephalitis, Hashimoto's thyroiditis, hemolytic anemia, Henoch-Schonlein purpura, hepatitis, herpes gestationis, hypogammaglobulinemia, idiopathic pulmonary fibrosis, Idiopathic thrombocytopenic purpura (ITP), IgA nephropathy, IgG4-related sclerosing disease, Immunoregulatory lipoproteins, inclusion body myositis, inflammatory bowel disorders, interstitial cystitis, juvenile arthritis, Juvenile diabetes (Type 1 diabetes), juvenile myositis, Kawasaki syndrome, keloid, Lambert-Eaton syndrome, leukocytoclastic vasculitis, lichen planus, lichen sclerosis, ligneous conjunctivitis, linear IgA disease, Lupus (SLE), Lyme disease, mediastinal fibrosis, Meniere's disease, microscopic polyangitis, Mixed connective tissue disease (MCTD), Mooren's ulcer, Mucha-Habermann disease, Multiple Sclerosis (MS), Myasthenia gravis, myelofibrosis, Myositis, narcolepsy, Neonatal Onset Multisystem Inflammatory Disease, nephrogenic systemic fibrosis, Neuromyelitis optica (Devic's), neutropenia, nonalcoholic fatty liver disease, nonalcoholic steatohepatitis (NASH), ocular-cicatricial pemphigold, optic neuritis, palindromic rheumatism, paraneoplastic cerebellar degeneration, Paroxysmal nocturnal hemoglobinuria (PNH), Parry Romberg syndrome, Pars planitis (peripheral uveitis), Parsonnage-Turner syndrome, Pediatric Autoimmune Neuropsychiatric Disorders Associated with Streptococcus (PANDAS), Pemphigus, Peripheral neuropathy, Perivenous encephalomyelitis, Pernicious anemia, Peyronie's disease, POEMS syndrome, polyarteritis nodosa, polymyalgia rhematica, polymyositis, postmyocardial infarction syndrome, postpericardiotomy syndrome, primary biliary cirrhosis, primary sclerosing cholangitis, progesterone dermatitis, progressive massive fibrosis, psoriasis, psoriatic arthritis, pure red cell aplasia, pyoderma gangrenosum, Raynauds phenomenon, reactic arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm and testicular autoimmunity, stiff person syndrome, subacute bacterial endocarditis, Susac's syndrome, sympathetic ophthalmia, systemic lupus erythematosus (SLE), Takayasu's arteritis, temporal arteritis, Thrombocytopenic purpura (TTP), Tolosa-Hunt syndrome, transverse myelitis, Tumor Necrosis Factor Receptor-associated Periodic Syndrome, Type 1 diabetes, Type I autoimmune polyglandular syndrome, Type II autoimmune polyglandular syndrome, Type III autoimmune polyglandular syndrome, ulcerative colitis, undifferentiated connective tissue disease, uveitis, vasculitis, vesiculobullous dermatosis, Vitiligo, and/or Granulomatosis with Polyangitis (GPA).

Embodiments of the disclosure include pharmaceutical preparations of cells, comprising any of the cells encompassed herein.

Other embodiments include methods of producing any cells encompassed herein. Such methods may comprise the step of reducing the expression of a polypeptide that is an immunogenic component involved in a pathological immune response. In some cases, the method further comprises the step of delivering a therapeutically effective amount of the cells to an individual at risk or having a medical condition.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### DETAILED DESCRIPTION

As used herein the specification, “a” or “an” may mean one or more. As used herein in the claim(s), when used in conjunction with the word “comprising”, the words “a” or “an” may mean one or more than one. As used herein “another” may mean at least a second or more. In specific embodiments, aspects of the disclosure may “consist essentially of” or “consist of” one or more sequences of the invention, for example. Some embodiments may consist of or consist essentially of one or more elements, method steps, and/or methods of the invention. It is contemplated that any method or composition described herein can be implemented with respect to any other method or composition described herein. The scope of the present application is not intended to be limited to the particular embodiments of the process, machine, manufacture, composition of matter, means, methods and steps described in the specification.

### I. Definitions

The term “engineered,” “modified,” or “genetically modified” as used herein refers to fibroblast cells that by the hand of man have been changed with respect to the expression and/or activity of at least one gene or other genetic element that is endogenous to the cell. In particular embodiments, the engineered fibroblasts are part of a combination of cells, materials, and/or biochemical factors used to improve and/or replace biological cells.

The term “immunogenic,” “immunogenic component,” “immunomodulation,” or “immunomodulatory,” refers to any process or gene product capable of modifying and/or regulating one or more immune functions.

The term “individual”, as used herein, refers to a human or animal that may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants. It is not intended that the term “individual” connote a need for medical treatment, therefore, an individual may voluntarily or involuntarily be part of experimentation whether clinical or in support of basic science studies. The term “subject” or “individual” refers to any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals.

The term “pharmaceutically” or “pharmacologically acceptable,” as used herein, refer to molecular entities and compositions that do not produce adverse, allergic, or other untoward reactions when administered to an animal or a human.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

“Therapeutic agent” means to have “therapeutic efficacy” in modulating angiogenesis and/or wound healing and an amount of the therapeutic is said to be a “angiogenic modulatory amount”, if administration of that amount of the therapeutic is sufficient to cause a significant modulation (i.e., increase or decrease) in angiogenic activity when administered to a subject (e.g., an animal model or human patient) needing modulation of angiogenesis.

The term “fibrosis” means the formation of excessive fibrous connective tissue in an organ or tissue. Fibrosis occurs in normal physiology to act as a deposit of connective tissue. In pathology, fibrosis can be used to describe an excess state of deposition of extracellular material and proteins that can result in scarring, thickening of the afflicted tissue, and interfere with the normal function of the tissue or organ.

As used herein, the term “therapeutically effective amount” is synonymous with “effective amount,” “therapeutically effective dose,” and/or “effective dose” and refers to the amount of compound that will elicit the biological, cosmetic or clinical response being sought by the practitioner in an individual in need thereof. As one example, an effective amount is the amount sufficient to reduce immunogenicity of a group of cells.

As used herein, the term “transplantation” refers to the process of taking living tissue or cells and implanting it in another part of the body or into another body.

“Treatment,” “treat,” or “treating” means a method of reducing the effects of a disease or condition. Treatment can also refer to a method of reducing the disease or condition itself rather than just the symptoms. The treatment can be any reduction from pre-treatment levels and can be but is not limited to the complete ablation of the disease, condition, or the symptoms of the disease or condition. Therefore, in the disclosed methods, treatment” can refer to a 10%, 20%, 30%, 40%, 50%, 60%, 70%, 80%, 90%, or 100% reduction in the severity of an established disease or the disease progression, including reduction in the severity of at least one symptom of the disease. For example, a disclosed method for reducing the immunogenicity of cells is considered to be a treatment if there is a detectable reduction in the immunogenicity of cells when compared to pre-treatment levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition. In specific embodiments, treatment refers to the lessening in severity or extent of at least one symptom and may alternatively or in addition refer to a delay in the onset of at least one symptom.

The term “administered” or “administering”, as used herein, refers to any method of providing a composition to an individual such that the composition has its intended effect on the patient. For example, one method of administering is by an indirect mechanism using a medical device such as, but not limited to a catheter, applicator gun, syringe etc. A second exemplary method of administering is by a direct mechanism such as, local tissue administration, oral ingestion, transdermal patch, topical, inhalation, suppository etc.

The term “delivering” or “delivered as used herein, refers to any method of providing a composition(s) to an individual such that the composition has its intended effect on the patient. For example, one method of administering is by an indirect mechanism using a medical device such as, but not limited to a catheter, applicator gun, syringe, etc. A second exemplary method of administering is by a direct mechanism such as, local tissue administration, oral ingestion, transdermal patch, topical, inhalation, suppository, etc.

As used herein, the terms “allostimulatory” and “alloreactive” refer to stimulation and reaction of the immune system in response to an allologous antigens, or “alloantigens” or cells expressing a dissimilar HLA haplotype.

As used herein, the term “autoimmunity” refers to the system of immune responses of an organism against its own healthy cells and tissues.

As used herein, “autologous” refers to tissues or cells that are derived or transferred from the same individual's body (i.e., autologous blood donation; an autologous bone marrow transplant).

As used herein, the term “autotransplantation” refers to the transplantation of organs, tissues, and/or cells from one part of the body in an individual to another part in the same individual, i.e., the donor and recipient are the same individual. Tissue transplanted by such “autologous” procedures is referred to as an autograft or autotransplant.

As used herein “allogeneic” refers to tissues or cells from another body that in a natural setting are immunologically incompatible or capable of being immunologically incompatible, although from one or more individuals of the same species.

“Cell culture” or “culture” or “cultured” refers to an artificial in vitro system containing viable cells, whether quiescent, senescent or (actively) dividing. In a cell culture, cells are grown and maintained at an appropriate temperature, typically a temperature of 37′C and under an atmosphere typically containing oxygen and CO₂. Culture conditions may vary widely for each cell type though, and variation of conditions for a particular cell type can result in different phenotypes being expressed.

### II. General Embodiments

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

A. Genetic Modification of Ex Vivo Cultured Cells

In some embodiments, there are methods of reducing the immunogenicity of a cell population to allow the population to be effective in a recipient individual. In specific embodiments, the population is subjected to genetic modification to reduce the expression of one or more immunogenic components, such as human leukocyte antigen (HLA)-A, HLA-B, HLA-C, HLA-DP, HLA-DQ, HLA-DR, HLA-B27, cluster of differentiation 40 (CD-40), CD80, CD86, CD11b, interleukin 12 (IL-12), lymphocyte function-associated antigen 1 (LFA-1), Interleukin adhesion molecule 1 (ICAM-1), platelet endothelial cell adhesion molecule (PECAM), epithelial cell adhesion molecule (EpCAM), V αβ 3 integrin, interferon gamma receptor, stimulator of interferon genes (STING), CIIT, transforming growth factor beta (TGF-β) receptors, members of the SMAD family and/or a combination thereof. In particular embodiments of the disclosure, the engineered fibroblasts have a partial or complete reduction in the expression in one or more immunogenic components for example via a recombinant expression vector operable in eukaryotic cells, and the expression of the immunogenic components may be regulated by a constitutive promoter or an inducible promoter or a tissue-specific promoter or a conditional promoter (such as hypoxia-inducible). In specific embodiments, the vector is a viral vector, such as a retrovirus, lentivirus, adenovirus, adeno-associated virus, or herpes simplex virus, or the vector is a non-viral vector, such as naked DNA or plasmid DNA or minicircle DNA. Agents for transfection include at least the following: Fas ligand, TGF-beta, IL-4, IL-10, HLA-G, indolamine 2,3 deoxygenase (IDO), galectin family members, Galectin 3, arginase, and/or IL-20, as examples.

In some embodiments, a population of cells are immortalized. Immortalization may be accomplished by introduction of one or more certain genes, including human telomerase reverse transcriptase (hTERT). The process of immortalization is known in the art and may involve utilization of various vectors to introduce genes into target cells, said vectors included are those based on viral vectors, such as retroviral (lentivirus for infecting dividing as well as non-dividing cells), foamy viruses (U.S. Pat. Nos. 5,624,820, 5,693,508, 5,665,577, 6,013,516 and 5,674,703; WO92/05266 and WO92/14829), adenovirus (U.S. Pat. Nos. 5,700,470, 5,731,172 and 5,928,944), adeno-associated virus (AAV) (U.S. Pat. No. 5,604,090), herpes simplex virus vectors (U.S. Pat. No. 5,501,979), cytomegalovirus (CMV) based vectors (U.S. Pat. No. 5,561,063), reovirus, rotavirus genomes, simian virus 40 (SV40) or papilloma virus (Cone et al., Proc. Natl. Acad. Sci. USA 81:6349 (1984); EUKARYOTIC VIRAL VECTORS, Cold Spring Harbor Laboratory, Gluzman ed., 1982; Sarver et al., Mol. Cell. Biol. 1:486 (1981); U.S. Pat. No. 5,719,054). Adenovirus efficiently infects slowly replicating and/or terminally differentiated cells and can be used to target slowly replicating and/or terminally differentiated cells. Simian virus 40 (SV40) and bovine papilloma virus (BPV) have the ability to replicate as extra-chromosomal elements (Eukaryotic Viral Vectors, Cold Spring Harbor Laboratory, Gluzman ed., 1982; Sarver et al., Mol. Cell. Biol. 1:486 (1981)). Additional viral vectors useful for expression include reovirus, parvovirus, Norwalk virus, coronaviruses, paramyxo and rhabdoviruses, togavirus (e.g., sindbis virus and semliki forest virus) and vesicular stomatitis virus (VSV) for introducing and directing expression of a polynucleotide or transgene in pluripotent stem cells or progeny thereof (e.g., differentiated cells). Vectors including a nucleic acid can be expressed when the nucleic acid is operably linked to an expression control element.

In one embodiment, disclosed is the use of a regulatory element that is operably linked to one or more elements of a CRISPR system so as to drive expression of the one or more elements of the CRISPR system, with the goal of manipulating DNA encoding for genes in fibroblasts in a manner that prevents fibroblasts from expressing human leukocyte antigen (HLA)-A, HLA-B, HLA-C, HLA-DP, HLA-DQ, HLA-DR, HLA-B27, cluster of differentiation 40 (CD-40), CD80, CD86, CD11b, interleukin 12 (IL-12), lymphocyte function-associated antigen 1 (LFA-1), Interleukin adhesion molecule 1 (ICAM-1), platelet endothelial cell adhesion molecule (PECAM), epithelial cell adhesion molecule (EpCAM), V αβ 3 integrin, interferon gamma receptor, stimulator of interferon genes (STING), CIIT, transforming growth factor beta (TGF-β) receptors, members of the SMAD family and/or a combination thereof.

CRISPRs, also known as SPIDRs (SPacer Interspersed Direct Repeats), constitute a family of DNA loci that are generally unique to a particular bacterial species. The CRISPR locus comprises a distinct class of interspersed short sequence repeats (SSRs) that were recognized in E. coli. The finding of SSRs was not specific to E. coli in that other groups have identified them in other bacteria such as in tuberculosis. The CRISPR loci differ from other SSRs by the structure of the repeats, which are called short regularly spaced repeats (SRSRs). Repeats of SRSRs are short elements that occur in clusters that are regularly spaced by unique intervening sequences with a substantially constant length. Although the repeat sequences are highly conserved between strains, the number of interspersed repeats and the sequences of the spacer regions typically differ from strain to strain. In the embodiment, endogenous CRISPR system is utilized to delete immunogenic components, formation of a CRISPR complex (which is made of a guide sequence hybridized to a target sequence and complexed with one or more Cas proteins) will cause cleavage of one or both strands in or near the target sequence. The tracr sequence may comprise or consist of all or a portion of a wild-type tracr sequence and may also form part of a CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of a CRISPR complex. When inducing gene editing in cells a Cas enzyme, a guide sequence linked to a tracr-mate sequence, and a tracr sequence could each be operably linked to separate regulatory elements on separate vectors. Useful vectors include viral constructs, which are well known in the art, in one particular embodiment lentiviral constructs are utilized. In one embodiment, two or more of the elements expressed from the same or different regulatory elements may be combined in a single vector, with one or more additional vectors providing any components of the CRISPR system that are not included in the first vector.

In one embodiment, a vector comprises one or more insertion sites, such as a restriction endonuclease recognition sequence. In some embodiments, one or more insertion sites are located upstream and/or downstream of one or more sequence elements of one or more vectors. In some embodiments, a vector comprises an insertion site upstream of a tracr mate sequence, and optionally downstream of a regulatory element operably linked to the tracr mate sequence, such that following insertion of a guide sequence into the insertion site and upon expression the guide sequence directs sequence-specific binding of a CRISPR complex to a target sequence in a eukaryotic cell. In some embodiments, a vector comprises two or more insertion sites, each insertion site being located between two tracr mate sequences so as to allow insertion of a guide sequence at each site. In such an arrangement, the two or more guide sequences may comprise two or more copies of a single guide sequence, two or more different guide sequences, or combinations of these. When multiple different guide sequences are used, a single expression construct may be used to target CRISPR activity to multiple different, corresponding target sequences within a cell.

In cases wherein recombination technology is employed, one or more types of cells are manipulated to harbor an expression vector that encodes a gene product of interest. A recombinant expression vector(s) can be introduced as one or more DNA molecules or constructs, where there may be at least one marker that will allow for selection of host cells that contain the vector(s). The vector(s) can be prepared in conventional ways, wherein the genes and regulatory regions may be isolated, as appropriate, ligated, cloned in an appropriate cloning host, and analyzed by sequencing or other convenient means. Particularly, using PCR, individual fragments including all or portions of a functional unit may be isolated, where in some cases one or more mutations may be introduced using “primer repair”, ligation, in vitro mutagenesis, etc. as appropriate. The vector(s) once completed and demonstrated to have the appropriate sequences may then be introduced into the host cell by any convenient means. The constructs may be integrated and packaged into non-replicating, defective viral genomes like lentivirus, Adenovirus, Adeno-associated virus (AAV), Herpes simplex virus (HSV), or others, including retroviral vectors, for infection or transduction into cells. The vector(s) may include viral sequences for transfection, if desired. Alternatively, the construct may be introduced by fusion, electroporation, biolistics, transfection, lipofection, or the like. The host cells may be grown and expanded in culture before introduction of the vector(s), followed by the appropriate treatment for introduction of the vector(s) and integration of the vector(s). The cells are then expanded and screened by virtue of a marker present in the construct. Various markers that may be used successfully include hprt, neomycin resistance, thymidine kinase, hygromycin resistance, etc.

Any of the genes encoding for immunogenic components described herein, or active portions thereof, may be cloned into mammalian expression constructs comprising one or more promoter sequences enabling expression in cells such as the CMV promoter [Artuc et al., Exp. Dermatol. 1995, 4:317-21]. Examples of suitable constructs include, but are not limited to pcDNA3, pcDNA3.1 (+/−), pGL3, PzeoSV2 (+/−), pDisplay, pEF/myc/cyto, pCMV/myc/cyto each of which is commercially available from Invitrogen Co. (www.invitrogen.com), or the pSH expression vector which enables a regulated polynucleotide expression in human foreskin cells [Ventura and Villa, 1993, Biochem. Biophys. Commun. 192: 867-9]. Examples of retroviral vector and packaging systems are those sold by Clontech, San Diego, Calif., USA, including Retro-X vectors pLNCX and pLXSN, which permit cloning into multiple cloning sites and the transgene is transcribed from CMV promoter. Vectors derived from Mo-MuLV are also included such as pBabe, where the transgene will be transcribed from the 5′LTR promoter. After completing plasmid transfection fibroblasts are harvested by a means allowing for detachment from tissue culture plates, for example, by trypsinization and transferred to either a 6-well (Nunc, Denmark) or a 24-well plate (Nunc) for proliferation. Approximately 3 days post-transfection, the cell media is changed to media allow for proliferation and expansion of engineered fibroblasts. One example is Neurobasal A (NBA) proliferation medium comprising Neurobasal-A (Invitrogen), 1% D-glucose (Sigma Aldrich), 1% Penicillin/Streptomycin/Glutamine (Invitrogen), 2% B27 supplement with Retinoic acid (Invitrogen), 0.2% EGF (Peprotech, USA), 0.08% FGF-2 (Peprotech), 0.2% Heparin (Sigma Aldrich, USA) and Valproic acid (Sigma-Aldrich). The media is then subsequently changed thrice weekly, and cells are re-plated regularly (for example, 2-8 times up to a maximum of weekly re-plating, becoming more regular as colonies began to develop) to remove non-reprogrammed cells until widespread colony formation is achieved.

The vector(s) may be introduced as a single DNA molecule encoding at least one agent (including one or more immunogenic polypeptides or functional fragments thereof) and optionally another polynucleotide (such as genes), or different DNA molecules having one or more polynucleotides (such as genes). The vector(s) may be introduced simultaneously or consecutively, each with the same or different markers. In an illustrative example, one vector would contain one or more agents.

Vector(s) comprising useful elements such as bacterial or yeast origins of replication, selectable and/or amplifiable markers, promoter/enhancer elements for expression in prokaryotes or eukaryotes, etc. that may be used to prepare stocks of vector DNAs and for carrying out transfections are well known in the art, and many are commercially available.

In certain embodiments, it is contemplated that RNAs or proteinaceous sequences may be co-expressed with other selected RNAs or proteinaceous sequences in the same host cell. Co-expression may be achieved by co-transfecting the host cell with two or more distinct recombinant vectors. Alternatively, a single recombinant vector may be constructed to include multiple distinct coding regions for RNAs, which could then be expressed in host cells transfected with the single vector.

In some situations, it may be desirable to kill the engineered fibroblast cells, such as when the object is to terminate the treatment, the cells become neoplastic, in research where the absence of the cells after their presence is of interest, and/or another event. For this purpose one can provide for the expression of certain gene products in which one can kill the engineered cells under controlled conditions, such as a suicide gene. Suicide genes are known in the art, e.g. the iCaspase9 system in which a modified form of caspase 9 is dimerizable with a small molecule, e.g. AP1903. See, e.g., Straathof et al., Blood 105:4247-4254 (2005).

B. Reducing Cellular Immunogenicity of Ex Vivo Cultured Cells

In some embodiments of the disclosure, there are methods and compositions related to reduction of the immunogenicity of a cell population, wherein the population is subjected to genetic modification to reduce the expression of polypeptides comprising human leukocyte antigen (HLA)-A, HLA-B, HLA-C, HLA-DP, HLA-DQ, HLA-DR, HLA-B27, cluster of differentiation 40 (CD-40), CD80, CD86, CD11b, interleukin 12 (IL-12), lymphocyte function-associated antigen 1 (LFA-1), Interleukin adhesion molecule 1 (ICAM-1), platelet endothelial cell adhesion molecule (PECAM), epithelial cell adhesion molecule (EpCAM), V αβ 3 integrin, interferon gamma receptor, stimulator of interferon genes (STING), CIIT, transforming growth factor beta (TGF-β) receptors, members of the SMAD family and/or a combination thereof.

In general embodiments, a population of cells is subjected to one or more compositions comprised of one or more particular media and/or one or more agents such that the composition(s) are capable of reducing the immunogenicity of the population of cells. In particular embodiments of the disclosure, methods are directed to a population of cells wherein the cells are fibroblasts of any type and the fibroblasts become modified such that they have reduced immunogenicity and may be utilized in a therapeutic capacity. In certain embodiments, the fibroblasts may be of any kind, including placenta, umbilical cord, foreskin, skin, omentum, adipose tissue, and/or bone marrow, or derivatives thereof, for example. In certain cases, methods of the disclosure are directed to autologous cells. In other cases, methods of the disclosure are directed to allogeneic cells. In some cases, methods of the disclosure are directed to xenogeneic cells.

Embodiments of the disclosure provide means of utilizing fibroblasts (or other types of cells, as noted above) as allogeneic therapeutic cells through modification of culture conditions in order to decrease immunogenicity of the fibroblasts. In one embodiment of the disclosure, fibroblasts are extracted from sources with lower immunogenicity (e.g. placental fibroblasts, etc.). In another embodiment, fibroblasts are cultured ex vivo and subjected to genetic modification which without being restricted to mechanism, has been demonstrated by the inventors to reduce immunogenicity. The reduction in immunogenicity may be exemplified by inhibiting the ability of the fibroblasts to evoke a pathological immune response.

In specific embodiments, the disclosure provides methods for assessment of immunogenicity to be performed, e.g., quantifying the ability to modulate mixed lymphocyte reaction. Mixed lymphocyte reactions are well known in the art. Typically, mixed lymphocyte reaction is performed by co-culturing fibroblasts (in this case, that have been genetically modified) together with allogeneic lymphocytes. In certain embodiments, parameters of the mixed lymphocyte reaction that indicate modulation in immunogenicity comprise T cell proliferation, cytokine secretion, and cytotoxicity. Methods for quantifying T cell proliferation, cytokine secretion, and cytotoxicity are well known in the art. In certain embodiments, modulation of immunogenicity can be determined by quantifying the secretion of one or more cytokines comprising TNF-alpha, Interferon gamma, interleukin (IL)-1, IL-2, IL-6, IL-7, IL-8, IL-12, IL-15, IL-17, IL-33, or a combination thereof.

In specific embodiments, the disclosure provides methods that pertain to the administration of cells with reduced immunogenicity to an individual in need thereof. The population of cells with reduced immunogenicity may be administered as desired. Depending upon the response desired, the manner of administration, the life of the cells, and/or the number of cells present, various protocols may be employed.

C. Treatment of Inflammatory-Associated or Autoimmune Disease

Inflammation in response to injury or in certain conditions represents the normal and healthy response of the body. However in a pathological setting, the immune system may attack the body's own cells or tissues and results in abnormal inflammation, which may lead to chronic pain, redness, swelling, stiffness, damage to normal tissues, and can progress to the development of inflammatory-associated diseases.

In one embodiment of the disclosure, effective amounts of the engineered fibroblasts as prepared in methods encompassed by the disclosure and are administered to an individual that has or is at risk of developing medical conditions known to be or associated with inflammatory or autoimmune diseases such as Acute Disseminated Encephalomyelitis, Acute necrotizing hemorrhagic leukoencephalitis, Addison's disease, adhesive capsulitis, Agammaglobulinemia, Alopecia areata, Amyloidosis, Ankylosing spondylitis, Anti-GBM nephritis, Antiphospholipid syndrome (APS), Anti-TBM nephritis, arthofibrosis, atrial fibrosis, autoimmune angioedema, autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune neutropenia, autoimmune oophoritis, autoimmune pancreatitis, autoimmune retinopathy, autoimmune thrombocytopenic purpura (ATP), autoimmune thyroid disease, autoimmune urticarial, axonal and neuronal neuropathies, Balo disease, Behcet's disease, benign mucosal pemphigold, bullous pemphigoid, cardiomyopathy, Castleman disease, Celiac disease, Chagas disease, chronic fatigue syndrome, Chronic inflammatory demyelinating polyneuropathy (CIDP), chronic Lyme disease, Chronic recurrent multifocal ostomyelitis (CRMO), Churg-Strauss syndrome, cicatricial pemphigold, cirrhosis, Cogans syndrome, cold agglutinin disease, congenital heart block, Coxsackie myocarditis, CREST disease, Crohn's disease, Cystic Fibrosis, deficiency of the interleukin-1 receptor antagonist, demyelinating neuropathies, dermatitis herpetiformis, dermatomyosis, Devic's disease (neuromyelitis optica), discoid lupus, Dressler's syndrome, Dupuytren's contracture, endometriosis, endomyocardial fibrosis, eosinophilic esophagitis, eosinophilic facsciitis, erythema nodosum, essential mixed cryoglobulinemia, Evans syndrome, experimental allergic encephalomyelitis, Familial Mediterranean Fever, Fibromyalgia Fibrosing alveolitis, Giant cell arteritis (temporal arteritis), giant cell myocarditis, glomerulonephritis, Glomerulonephritis, Goodpasture's syndrome, Graft-versus-host disease (GVHD), granulomatosus with polyangitis, Graves' disease, Guillain-Barre syndrome, Hashimoto's encephalitis, Hashimoto's thyroiditis, hemolytic anemia, Henoch-Schonlein purpura, hepatitis, herpes gestationis, hypogammaglobulinemia, idiopathic pulmonary fibrosis, Idiopathic thrombocytopenic purpura (ITP), IgA nephropathy, IgG4-related sclerosing disease, Immunoregulatory lipoproteins, inclusion body myositis, inflammatory bowel disorders, interstitial cystitis, juvenile arthritis, Juvenile diabetes (Type 1 diabetes), juvenile myositis, Kawasaki syndrome, keloid, Lambert-Eaton syndrome, leukocytoclastic vasculitis, lichen planus, lichen sclerosis, ligneous conjunctivitis, linear IgA disease, Lupus (SLE), Lyme disease, mediastinal fibrosis, Meniere's disease, microscopic polyangitis, Mixed connective tissue disease (MCTD), Mooren's ulcer, Mucha-Habermann disease, Multiple Sclerosis (MS), Myasthenia gravis, myelofibrosis, Myositis, narcolepsy, Neonatal Onset Multisystem Inflammatory Disease, nephrogenic systemic fibrosis, Neuromyelitis optica (Devic's), neutropenia, nonalcoholic fatty liver disease, nonalcoholic steatohepatitis (NASH), ocular-cicatricial pemphigold, optic neuritis, palindromic rheumatism, paraneoplastic cerebellar degeneration, Paroxysmal nocturnal hemoglobinuria (PNH), Parry Romberg syndrome, Pars planitis (peripheral uveitis), Parsonnage-Turner syndrome, Pediatric Autoimmune Neuropsychiatric Disorders Associated with Streptococcus (PANDAS), Pemphigus, Peripheral neuropathy, Perivenous encephalomyelitis, Pernicious anemia, Peyronie's disease, POEMS syndrome, polyarteritis nodosa, polymyalgia rhematica, polymyositis, postmyocardial infarction syndrome, postpericardiotomy syndrome, primary biliary cirrhosis, primary sclerosing cholangitis, progesterone dermatitis, progressive massive fibrosis, psoriasis, psoriatic arthritis, pure red cell aplasia, pyoderma gangrenosum, Raynauds phenomenon, reactic arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm and testicular autoimmunity, stiff person syndrome, subacute bacterial endocarditis, Susac's syndrome, sympathetic ophthalmia, systemic lupus erythematosus (SLE), Takayasu's arteritis, temporal arteritis, Thrombocytopenic purpura (TTP), Tolosa-Hunt syndrome, transverse myelitis, Tumor Necrosis Factor Receptor-associated Periodic Syndrome, Type 1 diabetes, Type I autoimmune polyglandular syndrome, Type II autoimmune polyglandular syndrome, Type III autoimmune polyglandular syndrome, ulcerative colitis, undifferentiated connective tissue disease, uveitis, vasculitis, vesiculobullous dermatosis, Vitiligo, and/or Granulomatosis with Polyangitis (GPA).

In some aspects, there is a method of treating inflammatory-associated or autoimmune diseases in an individual by providing to the individual an effective amount of a fibroblast cell population that has reduced immunogenicity, such that there is a reduction in the expression of the following immunogenic proteins: human leukocyte antigen (HLA)-A, HLA-B, HLA-C, HLA-DP, HLA-DQ, HLA-DR, HLA-B27, cluster of differentiation 40 (CD-40), CD80, CD86, CD11b, interleukin 12 (IL-12), lymphocyte function-associated antigen 1 (LFA-1), Interleukin adhesion molecule 1 (ICAM-1), platelet endothelial cell adhesion molecule (PECAM), epithelial cell adhesion molecule (EpCAM), V αβ 3 integrin, interferon gamma receptor, stimulator of interferon genes (STING), CIIT, transforming growth factor beta (TGF-β) receptors, members of the SMAD family and/or a combination thereof.

In other embodiments, the fibroblast cell therapy comprises fibroblast cells of any kind, originating from multiple regions of the mammalian body, including placenta, umbilical cord, foreskin, skin, omentum, adipose tissue, and/or bone marrow, or derivatives thereof, for example. In certain cases, methods of the disclosure are directed to autologous cells. In other cases, methods of the disclosure are directed to allogeneic cells. In some cases, methods of the disclosure are directed to xenogeneic cells.

Cell expansion for cells originating from any of the abovementioned tissues above may occur in clean room facilities purposely built for cell therapy manufacture and meeting GMP clean room classification. In a sterile class II biologic safety cabinet located in a class 10,000 clean production suite, cells were thawed under controlled conditions and washed in a 15 mL conical tube with 10 ML of complete DMEM-low glucose media (cDMEM) (GibcoBRL, Grand Island, N.Y.) supplemented with 20% Fetal Bovine Serum (Atlas) from dairy cattle confirmed to have no BSE % Fetal Bovine Serum specified to have Endotoxin level less than or equal to 100 EU/mL (with levels routinely less than or equal to 10 EU/mL) and hemoglobin level less than or equal to 30 mg/dl (levels routinely less than or equal to 25 mg/dl). The serum lot used is sequestered and one lot was used for all experiments. Cells are subsequently placed in a T-225 flask containing 45 mL of cDMEM and cultured for 24 hours at 37° C. at 5% CO₂ in a fully humidified atmosphere. This allowed the MSC to adhere. Non-adherent cells were washed off using cDMEM by gentle rinsing of the flask. This resulted in approximately 6 million cells per initiating T-225 flask. The cells of the first flask were then split into 4 flasks. Cells were grown for 4 days after which approximately 6 million cells per flask were present (24 million cells total). This scheme was repeated but cells were not expanded beyond 10 passages, and were then banked in 6 million cell aliquots in sealed vials for delivery. All processes in the generation, expansion, and product production were performed under conditions and testing that was compliant with current Good Manufacturing Processes and appropriate controls, as well as Guidances issued by the FDA in 1998 Guidance for Industry: Guidance for Human Somatic Cell Therapy and Gene Therapy; the 2008 Guidance for FDA Reviewers and Sponsors Content and Review of Chemistry, Manufacturing, and Control (CMC) Information for Human Somatic Cell Therapy Investigational New Drug Applications (INDs); and the 1993 FDA points-to-consider document for master cell banks were all followed for the generation of the cell products described. Donor cells are collected in sterile conditions, shipped to a contract manufacturing facility, assessed for lack of contamination and expanded. The expanded cells are stored in cryovials of approximately 6 million cells/vial, with approximately 100 vials per donor. At each step of the expansion quality control procedures were in place to ensure lack of contamination or abnormal cell growth.

In some aspects, the engineered fibroblasts treat an individual diagnosed with or at risk of developing an inflammatory-associated or autoimmune disease. In some cases, the individual would undergo a test, such as a blood test, to determine the presence and/or level (or absence) of one or more antibodies, proteins, inflammatory markers, and/or a combination thereof to diagnose an individual with (or determine the risk of) an inflammatory-associated or autoimmune disease. Additional bodily fluid test(s) to determine a level of inflammation in a given individual may include testing the fast insulin level, hemoglobin A1C, C reactive protein, serum ferritin, red blood cell width, or a combination thereof. Organ function assessment may also be analyzed to determine the degree of inflammation in a given individual. Read-outs for such organ function tests include, but are not limited to, liver function test, bromsulphalein test, Serum bilirubin test, liver enzyme tests, Blood ammonia test, basal metabolism rate, protein bound iodine test, thyroidal iodine clearance test, radioactive iodine excretion test, thyroid scan, triiodiothryonine levels test, T3 suppression test, serum thyroxine test, pancreatic enzymes test, lipase test, and/or a combination thereof.

An individual in need of immunomodulatory therapy may be provided an effective amount of engineered fibroblasts as described herein. The fibroblasts may be engineered to reduce expression of one or more immunogenic proteins that is reduced in comparison to a non-engineered fibroblast of the same type. The reduction in expression may be partial or to the extent that the expression may not be detectable. In some cases, the type of medical condition that renders the individual in need of immunomodulatory therapy may dictate which one or more genes are downregulated in expression, and this can be empirically determined by one of skill in the art.

D. Kits of the Disclosure

Any of the cellular and/or non-cellular compositions described herein or similar thereto may be comprised in a kit. In a non-limiting example, one or more reagents for use in methods for preparing cellular therapy may be comprised in a kit. Such reagents may include cells one or more growth factors, vector(s) one or more costimulatory factors, media, enzymes, buffers, nucleotides, salts, primers, and so forth. The kit components are provided in suitable container means. In specific embodiments, the kit comprises fibroblasts and/or primers and/or nucleic acids encoding one or more immunogenic proteins as described elsewhere herein.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, or may be a substrate with multiple compartments for a desired reaction.

Some components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile acceptable buffer and/or other diluent.

In specific embodiments, reagents and materials include primers for amplifying desired sequences, nucleotides, suitable buffers or buffer reagents, salt, and so forth, and in some cases the reagents include apparatus or reagents for isolation of a particular desired cell(s).

In particular embodiments, there are one or more apparatuses in the kit suitable for extracting one or more samples from an individual. The apparatus may be a syringe, fine needles, scalpel, and so forth.

### EXAMPLES

The following examples are included to demonstrate embodiments of the invention. It should be appreciated by those of skill in the art that the techniques disclosed in the examples that follow represent techniques discovered by the inventor to function well in the practice of the invention, and thus can be considered to constitute modes for its practice. However, those of skill in the art should, in light of the present disclosure, appreciate that many changes can be made in the specific embodiments which are disclosed and still obtain a like or similar result without departing from the spirit and scope of the invention.

### Example 1

### Cellular Transplantation Therapy for Immunomodulation

The present example concerns methods for immunomodulation of cellular therapy for an individual in need thereof.

An individual of any age may present with one or more signs of illness (e.g., fever, stomach discomfort, bleeding, fatigue, etc.) that may be associated with an autoimmune or inflammatory condition and seek diagnosis and treatment. Upon evaluation of the individual by a medical practitioner, it may be concluded that the individual presents one or more signs (e.g., joint pain and swelling, fatigue, skin problems, abdominal pain or digestive issues, recurring fever, swollen glands, etc.) of or has a history (e.g., genetic disposition) of inflammatory-associated or autoimmune disease including, but not limited to Acute Disseminated Encephalomyelitis, Acute necrotizing hemorrhagic leukoencephalitis, Addison's disease, adhesive capsulitis, Agammaglobulinemia, Alopecia areata, Amyloidosis, Ankylosing spondylitis, Anti-GBM nephritis, Antiphospholipid syndrome (APS), Anti-TBM nephritis, arthofibrosis, atrial fibrosis, autoimmune angioedema, autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune neutropenia, autoimmune oophoritis, autoimmune pancreatitis, autoimmune retinopathy, autoimmune thrombocytopenic purpura (ATP), autoimmune thyroid disease, autoimmune urticarial, axonal and neuronal neuropathies, Balo disease, Behcet's disease, benign mucosal pemphigold, bullous pemphigoid, cardiomyopathy, Castleman disease, Celiac disease, Chagas disease, chronic fatigue syndrome, Chronic inflammatory demyelinating polyneuropathy (CIDP), chronic Lyme disease, Chronic recurrent multifocal ostomyelitis (CRMO), Churg-Strauss syndrome, cicatricial pemphigold, cirrhosis, Cogans syndrome, cold agglutinin disease, congenital heart block, Coxsackie myocarditis, CREST disease, Crohn's disease, Cystic Fibrosis, deficiency of the interleukin-1 receptor antagonist, demyelinating neuropathies, dermatitis herpetiformis, dermatomyosis, Devic's disease (neuromyelitis optica), discoid lupus, Dressler's syndrome, Dupuytren's contracture, endometriosis, endomyocardial fibrosis, eosinophilic esophagitis, eosinophilic facsciitis, erythema nodosum, essential mixed cryoglobulinemia, Evans syndrome, experimental allergic encephalomyelitis, Familial Mediterranean Fever, Fibromyalgia Fibrosing alveolitis, Giant cell arteritis (temporal arteritis), giant cell myocarditis, glomerulonephritis, Glomerulonephritis, Goodpasture's syndrome, Graft-versus-host disease (GVHD), granulomatosus with polyangitis, Graves' disease, Guillain-Barre syndrome, Hashimoto's encephalitis, Hashimoto's thyroiditis, hemolytic anemia, Henoch-Schonlein purpura, hepatitis, herpes gestationis, hypogammaglobulinemia, idiopathic pulmonary fibrosis, Idiopathic thrombocytopenic purpura (ITP), IgA nephropathy, IgG4-related sclerosing disease, Immunoregulatory lipoproteins, inclusion body myositis, inflammatory bowel disorders, interstitial cystitis, juvenile arthritis, Juvenile diabetes (Type 1 diabetes), juvenile myositis, Kawasaki syndrome, keloid, Lambert-Eaton syndrome, leukocytoclastic vasculitis, lichen planus, lichen sclerosis, ligneous conjunctivitis, linear IgA disease, Lupus (SLE), Lyme disease, mediastinal fibrosis, Meniere's disease, microscopic polyangitis, Mixed connective tissue disease (MCTD), Mooren's ulcer, Mucha-Habermann disease, Multiple Sclerosis (MS), Myasthenia gravis, myelofibrosis, Myositis, narcolepsy, Neonatal Onset Multisystem Inflammatory Disease, nephrogenic systemic fibrosis, Neuromyelitis optica (Devic's), neutropenia, nonalcoholic fatty liver disease, nonalcoholic steatohepatitis (NASH), ocular-cicatricial pemphigold, optic neuritis, palindromic rheumatism, paraneoplastic cerebellar degeneration, Paroxysmal nocturnal hemoglobinuria (PNH), Parry Romberg syndrome, Pars planitis (peripheral uveitis), Parsonnage-Turner syndrome, Pediatric Autoimmune Neuropsychiatric Disorders Associated with Streptococcus (PANDAS), Pemphigus, Peripheral neuropathy, Perivenous encephalomyelitis, Pernicious anemia, Peyronie's disease, POEMS syndrome, polyarteritis nodosa, polymyalgia rhematica, polymyositis, postmyocardial infarction syndrome, postpericardiotomy syndrome, primary biliary cirrhosis, primary sclerosing cholangitis, progesterone dermatitis, progressive massive fibrosis, psoriasis, psoriatic arthritis, pure red cell aplasia, pyoderma gangrenosum, Raynauds phenomenon, reactic arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm and testicular autoimmunity, stiff person syndrome, subacute bacterial endocarditis, Susac's syndrome, sympathetic ophthalmia, systemic lupus erythematosus (SLE), Takayasu's arteritis, temporal arteritis, Thrombocytopenic purpura (TTP), Tolosa-Hunt syndrome, transverse myelitis, Tumor Necrosis Factor Receptor-associated Periodic Syndrome, Type 1 diabetes, Type I autoimmune polyglandular syndrome, Type II autoimmune polyglandular syndrome, Type III autoimmune polyglandular syndrome, ulcerative colitis, undifferentiated connective tissue disease, uveitis, vasculitis, vesiculobullous dermatosis, Vitiligo, and/or Granulomatosis with Polyangitis (GPA).

To diagnose an individual with an inflammatory-associated or autoimmune disease, the medical practitioner may order a physical exam of the patient along with a batch of tests involving the individual's bodily fluids (blood, urine, etc.). To diagnose an autoimmune disease, the individual may undergo a blood test to determine the presence, level, or absence of an anti-body, protein, inflammatory marker, and/or a combination thereof. Additional bodily fluid tests to determine a level of inflammation in a given individual may include testing the fast insulin level, hemoglobin A1C, C reactive protein, serum ferritin, red blood cell width, or a combination thereof. Organ function assessment may also be analyzed to determine the degree of inflammation in a given individual. Read-outs for such organ function tests include, but are not limited to, liver function test, bromsulphalein test, Serum bilirubin test, liver enzyme tests, Blood ammonia test, basal metabolism rate, protein bound iodine test, thyroidal iodine clearance test, radioactive iodine excretion test, thyroid scan, triiodiothryonine levels test, T3 suppression test, serum thyroxine test, pancreatic enzymes test, lipase test, and/or a combination thereof.

Following diagnosis with an inflammatory-associated or autoimmune disease, the individual may be required to take immunomodulatory medication such as, but not limited to, nonsteroidal anti-inflammatory drugs (NSAIDS such as aspirin, ibuprofen, or naproxen), corticosteroids, hydroxychloroquine, disease-modifying anti-rehumatic drugs (DMARDS), biologic drugs, and/or a combination thereof. The individual may require regular monitoring by a medical practitioner to investigate the progression of the diagnosed illness over time. The medical practitioner may require the individual to undergo physical exams and/or the aforementioned tests (for example, involving bodily fluids) over a course of minutes, hours, days, weeks, month, and/or years in order to properly monitor and treat the individual.

Following determination of a need for treatment or prevention of an inflammatory-associated or autoimmune disease, a therapeutically effective amount of engineered fibroblasts are provided to the individual. In some cases, the individual is provided multiple doses of the engineered fibroblasts, and the series of doses may or may not be of the same amount. The individual may be monitored over time to determine whether or not one or more symptoms have improved.

### Example 2

### Knockout of Genes in Fibroblasts

Foreskin fibroblasts were purchased from ATCC and grown in Optimem® media with 10% fetal calf serum. To cut out the HLA gene, the gene editing of beta 2 microglobulin (B2M) and class II MHC transactivator (CIITA) were knocked out using the CRISPR/Cas9 genome editing system; the pLentiCRISPR V2 plasmid (Addgene plasmid No. 52961). Two examples of guide sequences (single guide RNA 1 (sgRNA1), 5′-GAAAATGTTTCCTGACTCAG-3′ (SEQ ID NO:1); and sgRNA2, 5′-CCCCGGACGATATTGAACAA-3′; (SEQ ID NO:2)) flanking the start codon for human B2M and three guide sequences (sgRNA1, 5′-TCCTACACAATGCGTTGCC-3′(SEQ ID NO:3); sgRNA2, 5′-TGGCACACTGTGAGCTGCCT-3′(SEQ ID NO:4); and sgRNA2, 5′-GCCCCTAGAAGGTGGCTACC-3′; (SEQ ID NO:5)) flanking the start codon for human CIITA were designed with the freely available program from MIT (http://crispr.mit.edu/), and 2 oligos were synthesized for each guide sequence (Invitrogen Life Technologies). The pLentiCRISPR V2 was digested with BsmBI, and the annealed oligonucleotides were cloned into the vector; then, the pLentiCRISPR (with cloned sgRNA) was co-transfected into the 293FT Cell Line (Thermo Fisher Scientific, catalog No. R700-07) with packaging plasmids pCMV-VSV-G, pRSV-Rev, and pMDLg/pRRE (Addgene plasmids 8454, 12253, and 60488, respectively). The virus-containing supernatants were collected 48 hours after transfection and centrifuged at 1500×g and 4° C. for 10 minutes to pellet the cell debris; then, the supernatants were filtered through a 0.45-μm low-protein-binding membrane (Millipore) and used immediately to transduce the fibroblasts. After transduction, the fibroblasts were expanded on Matrigel-coated dishes for 4 days with puromycin (5 μg/mL) selection, and individual puromycin-resistant single-cell-derived colonies were harvested and expanded in culture. B2M and CIITA knockouts were verified via Sanger sequencing and Western blot analysis.

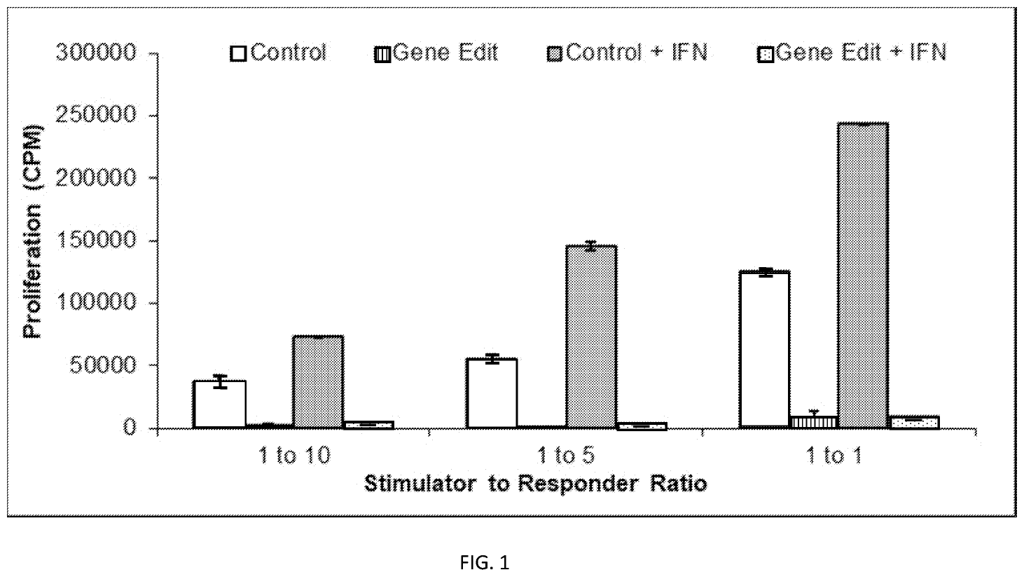

Fibroblasts lacking HLA by virtue of gene editing lacked ability to stimulate allogeneic T cells, either unstimulated or after stimulating with interferon gamma. Interferon gamma stimulation was achieved by culture for 24 hours in 100 international units of interferon gamma per ml. Fibroblasts where incubated with allogeneic peripheral blood mononuclear cells at the ratio of 1:10, 1:5, and 1:1. Proliferation was assessed by thymidine incorporation. As seen in FIG. 1, gene edited fibroblasts did not stimulate proliferation of allogeneic cells. This indicates lack of immunogenicity in fibroblasts subsequent to gene editing of HLA.

1. An engineered fibroblast cell comprising a reduction in the expression of a polynucleotide sequence encoding an immunogenic component selected from the group consisting of:
a) a human leukocyte antigen (HLA);
b) a costimulatory molecule;
c) an adhesion molecule;
d) a polypeptide associated with an increase in the expression of human leukocyte antigens;
e) a polypeptide associated with fibrosis; and
f) a combination thereof.

2. The engineered fibroblast cell of claim 1, wherein the HLA is HLA-A, HLA-B, HLA-C, HLA-DP, HLA-DQ, HLA-DR, HLA-B27, or a combination thereof.

3. The engineered fibroblast cell of claim 1, wherein the co-stimulatory molecule comprises cluster of differentiation 40 (CD-40), CD80, CD86, interleukin 12 (IL-12), or combination thereof.

4. The engineered fibroblast cell of claim 1, wherein the adhesion molecule comprises lymphocyte function-associated antigen 1 (LFA-1), Interleukin adhesion molecule 1 (ICAM-1), platelet endothelial cell adhesion molecule (PECAM), epithelial cell adhesion molecule (EpCAM), CD11b, V αβ 3 integrin, or a combination thereof.

5. The engineered fibroblast cell of claim 1, wherein the polypeptide associated with an increase in the expression of HLA comprises interferon gamma receptor, stimulator of interferon genes (STING), CIIT, or a combination thereof.

6. The engineered fibroblast cell of claim 1, wherein the polypeptide associated with fibrosis comprises transforming growth factor beta (TGF-β) receptors, members of the SMAD family, or a combination thereof.

7. The engineered fibroblast cell of claim 1, wherein the reduction in the expression of the polypeptide is mediated by CRISPR/Cas9, adenovirus, lentivirus, and/or adeno-associated virus and/or a combination thereof.

8. The engineered fibroblast cell of claim 1, wherein the engineered fibroblast expresses human telomerase reverse transcriptase (hTERT).

9. The engineered fibroblast cell of claim 8, wherein the engineered fibroblast cell originates from mammalian tissues.

10. The engineered fibroblast cell of claim 9, wherein the mammalian tissues are derived from a placenta, umbilical cord, foreskin, skin, omentum, adipose tissue, and/or bone marrow.

11. The engineered fibroblast cell of claim 9, wherein the mammalian tissues are derived from a human, primate, porcine, bovine, murine, canine, and/or feline.

12. A method of reducing an immune response to a fibroblast therapy in an individual, comprising the step of providing to the individual an effective amount of the fibroblast cells of claim 1.

13. The method of claim 12, wherein the fibroblast therapy is an immunomodulatory therapy.

14. The method of claim 12, wherein the individual has or is at risk for having Acute Disseminated Encephalomyelitis, Acute necrotizing hemorrhagic leukoencephalitis, Addison's disease, adhesive capsulitis, Agammaglobulinemia, Alopecia areata, Amyloidosis, Ankylosing spondylitis, Anti-GBM nephritis, Antiphospholipid syndrome (APS), Anti-TBM nephritis, arthofibrosis, atrial fibrosis, autoimmune angioedema, autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune neutropenia, autoimmune oophoritis, autoimmune pancreatitis, autoimmune retinopathy, autoimmune thrombocytopenic purpura (ATP), autoimmune thyroid disease, autoimmune urticarial, axonal and neuronal neuropathies, Balo disease, Behcet's disease, benign mucosal pemphigold, bullous pemphigoid, cardiomyopathy, Castleman disease, Celiac disease, Chagas disease, chronic fatigue syndrome, Chronic inflammatory demyelinating polyneuropathy (CIDP), chronic Lyme disease, Chronic recurrent multifocal ostomyelitis (CRMO), Churg-Strauss syndrome, cicatricial pemphigold, cirrhosis, Cogans syndrome, cold agglutinin disease, congenital heart block, Coxsackie myocarditis, CREST disease, Crohn's disease, Cystic Fibrosis, deficiency of the interleukin-1 receptor antagonist, demyelinating neuropathies, dermatitis herpetiformis, dermatomyosis, Devic's disease (neuromyelitis optica), discoid lupus, Dressler's syndrome, Dupuytren's contracture, endometriosis, endomyocardial fibrosis, eosinophilic esophagitis, eosinophilic facsciitis, erythema nodosum, essential mixed cryoglobulinemia, Evans syndrome, experimental allergic encephalomyelitis, Familial Mediterranean Fever, Fibromyalgia Fibrosing alveolitis, Giant cell arteritis (temporal arteritis), giant cell myocarditis, glomerulonephritis, Glomerulonephritis, Goodpasture's syndrome, Graft-versus-host disease (GVHD), granulomatosus with polyangitis, Graves' disease, Guillain-Barre syndrome, Hashimoto's encephalitis, Hashimoto's thyroiditis, hemolytic anemia, Henoch-Schonlein purpura, hepatitis, herpes gestationis, hypogammaglobulinemia, idiopathic pulmonary fibrosis, Idiopathic thrombocytopenic purpura (ITP), IgA nephropathy, IgG4-related sclerosing disease, Immunoregulatory lipoproteins, inclusion body myositis, inflammatory bowel disorders, interstitial cystitis, juvenile arthritis, Juvenile diabetes (Type 1 diabetes), juvenile myositis, Kawasaki syndrome, keloid, Lambert-Eaton syndrome, leukocytoclastic vasculitis, lichen planus, lichen sclerosis, ligneous conjunctivitis, linear IgA disease, Lupus (SLE), Lyme disease, mediastinal fibrosis, Meniere's disease, microscopic polyangitis, Mixed connective tissue disease (MCTD), Mooren's ulcer, Mucha-Habermann disease, Multiple Sclerosis (MS), Myasthenia gravis, myelofibrosis, Myositis, narcolepsy, Neonatal Onset Multisystem Inflammatory Disease, nephrogenic systemic fibrosis, Neuromyelitis optica (Devic's), neutropenia, nonalcoholic fatty liver disease, nonalcoholic steatohepatitis (NASH), ocular-cicatricial pemphigold, optic neuritis, palindromic rheumatism, paraneoplastic cerebellar degeneration, Paroxysmal nocturnal hemoglobinuria (PNH), Parry Romberg syndrome, Pars planitis (peripheral uveitis), Parsonnage-Turner syndrome, Pediatric Autoimmune Neuropsychiatric Disorders Associated with Streptococcus (PANDAS), Pemphigus, Peripheral neuropathy, Perivenous encephalomyelitis, Pernicious anemia, Peyronie's disease, POEMS syndrome, polyarteritis nodosa, polymyalgia rhematica, polymyositis, postmyocardial infarction syndrome, postpericardiotomy syndrome, primary biliary cirrhosis, primary sclerosing cholangitis, progesterone dermatitis, progressive massive fibrosis, psoriasis, psoriatic arthritis, pure red cell aplasia, pyoderma gangrenosum, Raynauds phenomenon, reactic arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm and testicular autoimmunity, stiff person syndrome, subacute bacterial endocarditis, Susac's syndrome, sympathetic ophthalmia, systemic lupus erythematosus (SLE), Takayasu's arteritis, temporal arteritis, Thrombocytopenic purpura (TTP), Tolosa-Hunt syndrome, transverse myelitis, Tumor Necrosis Factor Receptor-associated Periodic Syndrome, Type 1 diabetes, Type I autoimmune polyglandular syndrome, Type II autoimmune polyglandular syndrome, Type III autoimmune polyglandular syndrome, ulcerative colitis, undifferentiated connective tissue disease, uveitis, vasculitis, vesiculobullous dermatosis, Vitiligo, and/or Granulomatosis with Polyangitis (GPA).

15. The method of claim 12, wherein the individual is a mammal.

16. The method of claim 15, wherein the mammal is human, primate, porcine, bovine, murine, canine, or feline.

17. A method of reducing the immunogenicity of a cell population of claim 1, wherein the population comprises a reduction in the expression of one or more polypeptides encoded by a polynucleotide sequence.

18. The method of claim 17, wherein the cell population originates from mammalian tissues.

19. The method of claim 18, wherein the mammalian tissues are derived from a placenta, umbilical cord, foreskin, skin, omentum, adipose tissue, and/or bone marrow.

20. The method of claim 18, wherein the mammalian tissues are obtained from a human, primate, porcine, bovine, murine, canine, and/or feline.

21. The method of claim 17, wherein the fibroblast cells are cultured in a media.

22. The method of claim 21, wherein the media comprises Roswell Park Memorial Institute (RPMI-1640), Dublecco's Modified Essential Media (DMEM), Eagle's Modified Essential Media (EMEM), Optimem, Iscove's Media, or a combination thereof.

23. The method of claim 17, wherein the one or more polypeptides are immunogenic components.

24. The method of claim 23, wherein the immunogenic components comprise human leukocyte antigen (HLA)-A, HLA-B, HLA-C, HLA-DP, HLA-DQ, HLA-DR, HLA-B27, cluster of differentiation 40 (CD-40), CD80, CD86, CD11b, interleukin 12 (IL-12), lymphocyte function-associated antigen 1 (LFA-1), Interleukin adhesion molecule 1 (ICAM-1), platelet endothelial cell adhesion molecule (PECAM), epithelial cell adhesion molecule (EpCAM), V αβ 3 integrin, interferon gamma receptor, stimulator of interferon genes (STING), CIIT, transforming growth factor beta (TGF-β) receptors, members of the SMAD family and/or a combination thereof.

25. The method of claim 24, wherein the polypeptide expression is reduced by CRISPR/Cas9, adenovirus, lentivirus, and/or adeno-associated virus and/or a combination thereof.

26. A method of treating an autoimmune or inflammatory condition in an individual comprising the step of providing to the individual a therapeutically effective amount of cells of claim 1.

27. The method of claim 26, wherein the cells are immortalized fibroblast cells.

28. The method of claim 26, wherein the cells express hTERT.

29. The method of claim 26, wherein the individual has or is at risk of having Acute Disseminated Encephalomyelitis, Acute necrotizing hemorrhagic leukoencephalitis, Addison's disease, adhesive capsulitis, Agammaglobulinemia, Alopecia areata, Amyloidosis, Ankylosing spondylitis, Anti-GBM nephritis, Antiphospholipid syndrome (APS), Anti-TBM nephritis, arthofibrosis, atrial fibrosis, autoimmune angioedema, autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune neutropenia, autoimmune oophoritis, autoimmune pancreatitis, autoimmune retinopathy, autoimmune thrombocytopenic purpura (ATP), autoimmune thyroid disease, autoimmune urticarial, axonal and neuronal neuropathies, Balo disease, Behcet's disease, benign mucosal pemphigold, bullous pemphigoid, cardiomyopathy, Castleman disease, Celiac disease, Chagas disease, chronic fatigue syndrome, Chronic inflammatory demyelinating polyneuropathy (CIDP), chronic Lyme disease, Chronic recurrent multifocal ostomyelitis (CRMO), Churg-Strauss syndrome, cicatricial pemphigold, cirrhosis, Cogans syndrome, cold agglutinin disease, congenital heart block, Coxsackie myocarditis, CREST disease, Crohn's disease, Cystic Fibrosis, deficiency of the interleukin-1 receptor antagonist, demyelinating neuropathies, dermatitis herpetiformis, dermatomyosis, Devic's disease (neuromyelitis optica), discoid lupus, Dressler's syndrome, Dupuytren's contracture, endometriosis, endomyocardial fibrosis, eosinophilic esophagitis, eosinophilic facsciitis, erythema nodosum, essential mixed cryoglobulinemia, Evans syndrome, experimental allergic encephalomyelitis, Familial Mediterranean Fever, Fibromyalgia Fibrosing alveolitis, Giant cell arteritis (temporal arteritis), giant cell myocarditis, glomerulonephritis, Glomerulonephritis, Goodpasture's syndrome, Graft-versus-host disease (GVHD), granulomatosus with polyangitis, Graves' disease, Guillain-Barre syndrome, Hashimoto's encephalitis, Hashimoto's thyroiditis, hemolytic anemia, Henoch-Schonlein purpura, hepatitis, herpes gestationis, hypogammaglobulinemia, idiopathic pulmonary fibrosis, Idiopathic thrombocytopenic purpura (ITP), IgA nephropathy, IgG4-related sclerosing disease, Immunoregulatory lipoproteins, inclusion body myositis, inflammatory bowel disorders, interstitial cystitis, juvenile arthritis, Juvenile diabetes (Type 1 diabetes), juvenile myositis, Kawasaki syndrome, keloid, Lambert-Eaton syndrome, leukocytoclastic vasculitis, lichen planus, lichen sclerosis, ligneous conjunctivitis, linear IgA disease, Lupus (SLE), Lyme disease, mediastinal fibrosis, Meniere's disease, microscopic polyangitis, Mixed connective tissue disease (MCTD), Mooren's ulcer, Mucha-Habermann disease, Multiple Sclerosis (MS), Myasthenia gravis, myelofibrosis, Myositis, narcolepsy, Neonatal Onset Multisystem Inflammatory Disease, nephrogenic systemic fibrosis, Neuromyelitis optica (Devic's), neutropenia, nonalcoholic fatty liver disease, nonalcoholic steatohepatitis (NASH), ocular-cicatricial pemphigold, optic neuritis, palindromic rheumatism, paraneoplastic cerebellar degeneration, Paroxysmal nocturnal hemoglobinuria (PNH), Parry Romberg syndrome, Pars planitis (peripheral uveitis), Parsonnage-Turner syndrome, Pediatric Autoimmune Neuropsychiatric Disorders Associated with Streptococcus (PANDAS), Pemphigus, Peripheral neuropathy, Perivenous encephalomyelitis, Pernicious anemia, Peyronie's disease, POEMS syndrome, polyarteritis nodosa, polymyalgia rhematica, polymyositis, postmyocardial infarction syndrome, postpericardiotomy syndrome, primary biliary cirrhosis, primary sclerosing cholangitis, progesterone dermatitis, progressive massive fibrosis, psoriasis, psoriatic arthritis, pure red cell aplasia, pyoderma gangrenosum, Raynauds phenomenon, reactic arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm and testicular autoimmunity, stiff person syndrome, subacute bacterial endocarditis, Susac's syndrome, sympathetic ophthalmia, systemic lupus erythematosus (SLE), Takayasu's arteritis, temporal arteritis, Thrombocytopenic purpura (TTP), Tolosa-Hunt syndrome, transverse myelitis, Tumor Necrosis Factor Receptor-associated Periodic Syndrome, Type 1 diabetes, Type I autoimmune polyglandular syndrome, Type II autoimmune polyglandular syndrome, Type III autoimmune polyglandular syndrome, ulcerative colitis, undifferentiated connective tissue disease, uveitis, vasculitis, vesiculobullous dermatosis, Vitiligo, and/or Granulomatosis with Polyangitis (GPA).

30. A pharmaceutical preparation of cells, comprising the cells of claim 1 in a pharmaceutically acceptable carrier.

31. A method of producing the cells of claim 1, comprising the step of reducing in one or more fibroblast cells the expression of a polypeptide that is an immunogenic component involved in a pathological immune response.

32. The method of claim 31, further comprising the step of delivering a therapeutically effective amount of the cells to an individual at risk or having a medical condition.
